(19) United States
(12) Patent Application Publication
(10) Pub. No.: US 2024/0298882 A1
(43) Pub. Date: Sep. 12, 2024

(54) A SYSTEM FOR POSITIONING A MEDICAL TOOL

(71) Applicant: UCL BUSINESS LTD, London Greater London (GB)

(72) Inventors: Lukas LINDENROTH, London Greater London (GB); Joseph MANJALY, London Greater London (GB); Sophia BANO, London Greater London (GB); Agostino STILLI, London Greater London (GB); George DWYER, London Greater London (GB); Danail STOYANOV, London Greater London (GB)

(21) Appl. No.: 18/258,414

(22) PCT Filed: Dec. 15, 2021
(86) PCT No.: PCT/GB2021/053296

§ 371 Date: Jun. 20, 2023

(30) Foreign Application Priority Data
Dec. 21, 2020 (GB) 2020301.4

(51) Int. Cl. A61B 1/00; A61B 34/20; A61F 11/20

(52) U.S. Cl. CPC A61B 1/00154; A61F 11/20; A61B 2034/2051; A61B 2034/2057

(57) ABSTRACT

The present invention relates to a system 10 for positioning a medical tool within a lumen. The system 10 comprises a positioning device 11 and a control system 12. The positioning device 11 comprises a cylindrical body 110, configured to receive the medical tool, and an actuator 120 configured to apply a force to an internal wall of the lumen to move the cylindrical body 110 in a radial direction within the lumen. The control system 12 is configured to: receive an input signal indicative of a target position of the cylindrical body 110, and provide an output signal to control the actuator 120 to move the cylindrical body 110 to adopt the target position. The system 10 may comprise a camera and utilise image recognition to identify a target location in an image captured by the camera to determine the target position of the cylindrical body 110. The system 10 may be configured such that the cylindrical body 110 is automatically moved to the target position. The system 10 may also be configured to compensate for unintended movement of the cylindrical body 110 away from the target position. The system 10 may be particularly applicable to positioning a needle within an ear canal to perform an intratympanic injection.

### TECHNICAL FIELD

The present invention relates to a system for positioning a medical tool within a lumen.

### BACKGROUND

A number of medical procedures require the insertion of a medical tool into a lumen of a patient. For example, an intratympanic steroid injection requires insertion of a needle into the ear canal of a patient. In such procedures, controlled positioning of the medical tool within the lumen is required. In the example of an intratympanic steroid injection, the needle must be positioned to pierce the posteroinferior quadrant of the tympanic membrane such that a steroid can be injected to perfuse via the round window of the cochlea. At the same time, the needle must be stabilised within the ear canal so as not to cause discomfort or damage to the patient.

### SUMMARY OF INVENTION

A first aspect of the invention provides a system for positioning a medical tool within a lumen. The system comprises a positioning device and a control system. The positioning device comprises a cylindrical body, configured to receive the medical tool, and an actuator configured to apply a force to an internal wall of the lumen to move the cylindrical body in a radial direction within the lumen. The control system is configured to: receive an input signal indicative of a target position of the cylindrical body, and provide an output signal to control the actuator to move the cylindrical body to adopt the target position.

Applying a force to an internal wall of the lumen has the effect of both adjusting a radial position of the cylindrical body and stabilising the cylindrical body against the internal wall of the lumen. The actuator may be configured such that when actuated, the actuator supports the weight of the cylindrical body within the lumen.

The actuator may not be configured to apply a force to an internal wall of the lumen to move the cylindrical body in a longitudinal direction along the lumen. The system may not comprise any actuator configured to move the cylindrical body in a longitudinal direction along the lumen.

The target position may comprise a radial position of a longitudinal axis of the cylindrical body relative to a longitudinal axis of the lumen. The target position may comprise an angular orientation of a longitudinal axis of the cylindrical body relative to a longitudinal axis of the lumen.

The system may be configured to autonomously move the cylindrical body to adopt the target position. This reduces the extent of the human-operator input required to position the medical tool, thereby reducing the potential for error in positioning the medical tool. The control system may be configured to automatically obtain the input signal indicative of a target position of the cylindrical body. The control system may be configured to automatically provide the output signal to control the actuator to move the cylindrical body to adopt the target position.

The system may comprise a camera or optical fibres. The control system may be configured to process an image captured by the camera or optical fibres to produce the input signal indicative of the target position. The image may comprise an image of a sequence of images captured by the camera or optical fibres within a period of time. The image may comprise a frame of a video captured by the camera or optical fibres. The control system may be configured to process a plurality of images to produce the input signal indicative of the target position. Each image of the plurality of images may comprise an image of a sequence of images captured by the camera or optical fibres within a period of time, or a frame of a video captured by the camera or optical fibres. The control system may be configured to process the image to identify a target location in the image and produce the input signal indicative of the target position of the cylindrical body in dependence on the target location.

Where the control system is configured to process a plurality of images, the control system may be configured to do so to determine a spatio-temporal variation of the target location within a field of view of the plurality of images. The control system may be configured to process the image using an artificial neural network trained to identify a target location in the image and produce the input signal indicative of the target position of the cylindrical body in dependence on the target location in the image. The artificial neural network may be trained to perform semantic segmentation to label one or more pixels of the image. The artificial neural network may comprise a deep neural network.

The deep neural network may comprise a spatial pyramid pooling module and an encoder-decoder structure.

The control system may be configured to receive a further input signal indicative of a deviation of the cylindrical body from the target position. The control system may be configured to provide a further output signal to control the actuator to move the cylindrical body to adopt the target position in response to the further input signal. The system may therefore automatically reposition the cylindrical body of the positioning device in response to a deviation from the target position. For example, an unintended movement from the patient or a human operator of the system may cause the cylindrical body to deviate from the target position. The system can then automatically reposition the cylindrical body accordingly. Alternatively, or in addition, the control system may be configured to notify a human operator of the system in response to receiving the further input. The human operator may then decide whether or not corrective action is required.

The cylindrical body may comprise a core. The cylindrical body may comprise an outer membrane arranged on an external surface of the core. One or both of the core and the membrane may be formed from a compliant material. The compliant material may comprise silicone. The core may comprise a stiffness which is greater than a stiffness of the membrane. The diameter of the cylindrical body may be in the range of 3 mm to 5 mm, for example 4 mm. The length of the cylindrical body may be in the range of 6 mm to 14 mm, for example 10 mm. It will be appreciated that there are multiple applications of the invention, and that suitable dimensions for the cylindrical body may be selected in dependence on a particular application.

The actuator may comprise an inflatable actuator. Where the cylindrical body comprises an outer membrane, the actuator may comprise a portion of the outer membrane. The inflatable actuator may be more reliable, for example, than an actuator which utilises multiple co-operating moving parts which may be subject to mechanical failure. The inflatable actuator also inherently facilitates the use of compliant materials which are particularly suited to insertion in a lumen.

The system may comprise a positive displacement pump configured to deliver a fluid to the inflatable actuator to inflate the actuator. The positive displacement pump may comprise a cylinder and a piston received within the cylinder. The output signal to control the actuator may be indicative of a target volume to inflate the inflatable actuator to. The inflatable actuator may be inflatable with a liquid. The liquid may be deionised water.

The force applied by the actuator may be offset from the centre of the cylindrical body. This may enable the cylindrical body to be rotated about the centre of the cylindrical body, i.e. tilted with respect to the longitudinal axis of the lumen such that the cylindrical body is rotated about an axis perpendicular to the longitudinal axis of the lumen. This may provide for more accurate positioning of the cylindrical body.

The positioning device may comprise a plurality of actuators. Each actuator may be configured to apply a radial force to an internal wall of the lumen to move the cylindrical body in a different radial direction. The plurality of actuators may be configured to move the cylindrical body in two or more degrees of freedom.

The positioning device may comprise two or more actuators configured to move the cylindrical body about a pitch axis and/or a yaw axis. The positioning device may comprise a first actuator located at a first longitudinal position of the cylindrical body and a second actuator located at a second longitudinal position of the cylindrical body. The positioning device may comprise a first plurality of actuators located at a first longitudinal position of the cylindrical body and a second plurality of actuators located at a second longitudinal position of the cylindrical body.

The output signal to control the actuator may comprise a pressure to be applied by the actuator to the internal wall of the lumen. This may ensure sufficient pressure is applied to move the cylindrical body by the required amount while at the same time ensuring the pressure applied to the internal wall of the lumen is not harmful.

The medical tool may comprise a needle. The system may comprise the needle. The needle may be configured to deliver a medication to be injected. The cylindrical body of the positioning device may comprise a longitudinal bore for receiving the needle. The diameter of the longitudinal bore may be selected to provide a clearance fit with the needle. This may allow the needle to pass through the longitudinal bore while limiting radial movement of the needle with respect to the cylindrical body of the positioning device. In use, the needle may be moved, for example pushed by hand by a human operator, through the longitudinal bore relative to the cylindrical body of the positioning device. The system may be operated to fix the position of the cylindrical body within a lumen while the needle is moved through the longitudinal bore relative to the cylindrical body.

It will be appreciated that the diameter of the needle, and hence the diameter of the longitudinal bore, will vary depending on the particular use of the system. For example, in the case of intratympanic injections, a 27G needle may be used. The diameter of the longitudinal bore may be selected to provide a clearance fit with a 27G needle.

The cylindrical body may comprise a disposable element. The positioning device may comprise a reusable insert. The reusable insert may comprise the camera or optical fibres, the longitudinal bore for receiving the needle, and a plurality of inflation tubes. The reusable insert may comprise a main body comprising the longitudinal bore for receiving the needle and a housing for the camera. The cylindrical body may comprise a longitudinal bore configured to receive the main body of the reusable insert and a plurality of inflation channels configured to receive the inflation tubes.

In use, the disposable element providing the cylindrical body can be disposed of after use and a new, sterilised disposable element can be installed on the reusable insert for the next use. This provides advantages with respect to hygiene. Using the reusable insert with the camera incorporated therein allows for a fixed calibration between the camera and the longitudinal axis of the positioning device, which greatly simplifies deploying the system in clinical settings.

The lumen may comprise an ear canal. The target position of the cylindrical body may be aligned with the round window of a cochlea. The target position of the cylindrical body may be such that the needle is positioned to pierce a tympanic membrane at a location that enables a medication to be injected, via the needle, behind the tympanic membrane to perfuse via the round window of a cochlea. Where the system comprises a camera or optical fibres, and the control system is configured to process an image captured by the camera or optical fibres, the image may comprise an image of the tympanic membrane. Where the control system is configured to process the image to identify a target location in the image, the target location may be within the posteroinferior quadrant of the tympanic membrane. The target location may be the centre of the posteroinferior quadrant of the tympanic membrane.

It will be appreciated that the processing of the image by the control system may not be completely accurate. For example, the location of the centre of the posteroinferior quadrant of the tympanic membrane identified in the image may not be the actual location of the centre of the posteroinferior quadrant of the tympanic membrane. However, the input signal indicative of the target positon of the cylindrical body produced in dependence on the target location in the image will be sufficiently accurate so as to sufficiently position the medical tool. For example, the target positon of the cylindrical body produced in dependence on the centre of the posteroinferior quadrant of the tympanic membrane as identified by the processing of the image will enable the needle to be positioned such that the tympanic membrane can be pierced at a location that enables a medication to be injected, via the needle, behind the tympanic membrane to perfuse via the round window of the cochlea.

A second aspect of the invention provides a method of positioning a medical tool within a lumen. The method comprises: inserting a positioning device into the lumen, the positioning device comprising a cylindrical body configured to receive the medical tool, and an actuator configured to apply a force to an internal wall of the lumen to move the cylindrical body in a radial direction within the lumen; receiving an input signal, at a control system, indicative of a target position of the cylindrical body; and providing an output signal from the control system to control the actuator to move the cylindrical body to adopt the target position.

The method may comprise providing an output signal from the control system to control the actuator to stabilise the positioning device within the lumen and retain the longitudinal position of the positioning device within the lumen.

The method may comprise providing an output signal from the control system to control the actuator to maintain the cylindrical body at the target position.

The method may comprise receiving a further input signal indicative of a deviation of the cylindrical body from the target position. The method may comprise providing a further output signal to control the actuator to move the cylindrical body to adopt the target position in response to the further input signal. The method may comprise automatically repositioning the cylindrical body of the positioning device in response to a deviation from the target position. A deviation from the target position may be caused, for example, by an unintended movement from a patient or a human operator.

The medical tool may comprise a needle. The cylindrical body of the positioning device may comprise a longitudinal bore for receiving the needle.

The lumen may comprise an ear canal. The target position of the cylindrical body may be aligned with the round window of a cochlea. The target position of the cylindrical body may be such that the needle is positioned to pierce a tympanic membrane at a location that enables a medication to be injected, via the needle, behind the tympanic membrane to perfuse via the round window of a cochlea.

The method may comprise moving the needle through the longitudinal bore relative to the cylindrical body of the positioning device. The method may comprise moving the needle through the longitudinal bore relative to the cylindrical body of the positioning device while providing an output signal from the control system to control the actuator to maintain the cylindrical body at the target position. The method may comprise moving the needle through the longitudinal bore relative to the cylindrical body of the positioning device while the cylindrical body is fixed at the target position within the lumen.

Moving the needle through the longitudinal bore relative to the cylindrical body of the positioning device may comprise pushing the needle by hand by a human operator.

The positioning device of the second aspect of the invention may comprise any feature of the positioning device of the first aspect of the invention.

The control system of the second aspect of the invention may comprise any feature of the control system of the first aspect of the invention.

A third aspect of the invention provides a system for performing an intratympanic injection. The system comprises a positioning device configured to be received within an ear canal, a needle configured to deliver a medication to be injected, and a control system. The positioning device comprises a cylindrical body, comprising a longitudinal bore configured to receive the needle and an actuator configured to apply a force to an internal wall of an ear canal to move the cylindrical body in a radial direction within the ear canal. The control system is configured to: receive an input signal indicative of a target position of the cylindrical body, and provide an output signal to control the actuator to move the cylindrical body to adopt the target position.

The diameter of the longitudinal bore may be selected to provide a clearance fit with the needle. This may allow the needle to pass through the longitudinal bore while limiting radial movement of the needle with respect to the cylindrical body of the positioning device. In use, the needle may be moved, for example pushed by hand by a human operator, through the longitudinal bore relative to the cylindrical body of the positioning device. The system may be operated to fix the position of the cylindrical body within an ear canal while the needle is moved through the longitudinal bore relative to the cylindrical body.

The positioning device of the third aspect of the invention may comprise any feature of the positioning device of the first aspect of the invention.

The control system of the third aspect of the invention may comprise any feature of the control system of the first aspect of the invention.

### BRIEF DESCRIPTION OF THE DRAWINGS

Embodiments of the invention will now be described, by way of example only, with reference to the accompanying drawings:

FIG. 1 shows a system for positioning a medical tool within a lumen according to an aspect of the invention;

FIG. 2a shows an isometric schematic view of a positioning device of a system according to an embodiment of the invention;

FIG. 2b shows a front schematic view of the positioning device of FIG. 2a;

FIG. 2c shows a rear schematic view of the positioning device of FIG. 2a;

FIG. 3 shows a schematic representation of the positioning device of FIGS. 2a to 2c in use within an ear canal;

FIGS. 4a-c show a schematic representation of a moulding apparatus for producing the positioning device of FIGS. 2a to 2c;

FIG. 5 shows a system according to an embodiment of the invention;

FIGS. 6a to 6h illustrate schematically how the positioning device of FIGS. 2a to 2c can be maneuvered within an ear canal in one illustrative example;

FIG. 7 shows a schematic representation of an ear;

FIG. 8 shows a schematic representation of the tympanic membrane;

FIGS. 9a to 9d illustrate an image processing process used in an embodiment of the invention;

FIGS. 10a to 10c show results of the process of FIGS. 9a to 9d applied to an image of a human tympanic membrane;

FIGS. 11a to 11c show results of the process of FIGS. 9a to 9d applied to a further image of a human tympanic membrane;

FIGS. 12a to 12c show results of the process of FIGS. 9a to 9d applied to an image of a phantom tympanic membrane;

FIGS. 13a to 13c show results of the process of FIGS. 9a to 9d applied to a further image of a phantom tympanic membrane;

FIGS. 14a and 14b show an experimental set-up used measure the radial displacement of an actuator;

FIG. 15 shows a plot of mean actuator displacement ‘q’ in mm and standard deviation against percentage of a maximum inflation volume ‘V’ of an inflatable actuator for three prototype positioning devices;

FIG. 16a shows tracked linear translation of a cylindrical body of a prototype positioning device;

FIG. 16b shows tracked rotation about a centre of a cylindrical body of a prototype positioning device;

FIG. 16c shows tracked circular trajectories in translation of a cylindrical body of a prototype positioning device;

FIG. 16d shows tracked circular trajectories in rotation of a cylindrical body of a prototype positioning device;

FIG. 17 shows an experimental set-up used to test motion-compensation capabilities of a system of an embodiment of the invention;

FIG. 18a shows tracked translational motion of a prototype positioning device with inflatable actuators inflated to four different volumes;

FIG. 18b shows mean peak displacement of an inflatable actuator of three different prototype poisoning devices inflated to four different volumes;

FIG. 19a shows an experimental set-up used to test image recognition capabilities of a system of an embodiment of the invention;

FIGS. 19b and 19c show a phantom ear canal within which an image of a tympanic membrane is positioned;

FIG. 20a shows the standard deviation ‘o’ of a target location identified using the experimental set-up of FIG. 19a for each of the three test images at three different distances between the camera and the test image;

FIG. 20b shows the standard deviation ‘o’ of a target location identified using the experimental set-up of FIG. 19a for each of the three test images at three different angles of incidence between the camera and the test image;

FIG. 20c shows a plot of target locations identified by a prototype system as the cylindrical body of the positioning device of the system is moved in a circular motion;

FIGS. 21a and 21b show a schematic view of a positioning device 11 of a system according to another embodiment of the invention; and

FIG. 22 shows a schematic representation of a moulding apparatus for producing a cylindrical body of the positioning device of FIGS. 21a and 21b.

### DETAILED DESCRIPTION

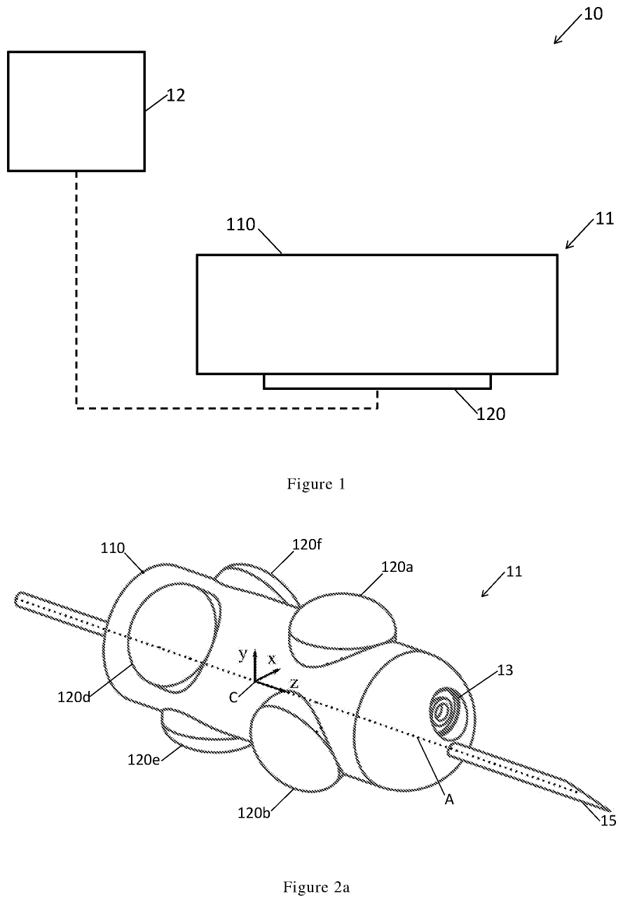

FIG. 1 shows a system 10, for positioning a medical tool within a lumen, according to an aspect of the invention. The system 10 comprises a positioning device 11 and a control system 12. The positioning device 11 comprises a cylindrical body 110, configured to receive the medical tool, and an actuator 120 configured to apply a force to an internal wall of the lumen to move the cylindrical body 110 in a radial direction within the lumen. The control system 12 is configured to: receive an input signal indicative of a target position of the cylindrical body 110, and provide an output signal to control the actuator 120 to move the cylindrical body 110 to adopt the target position.

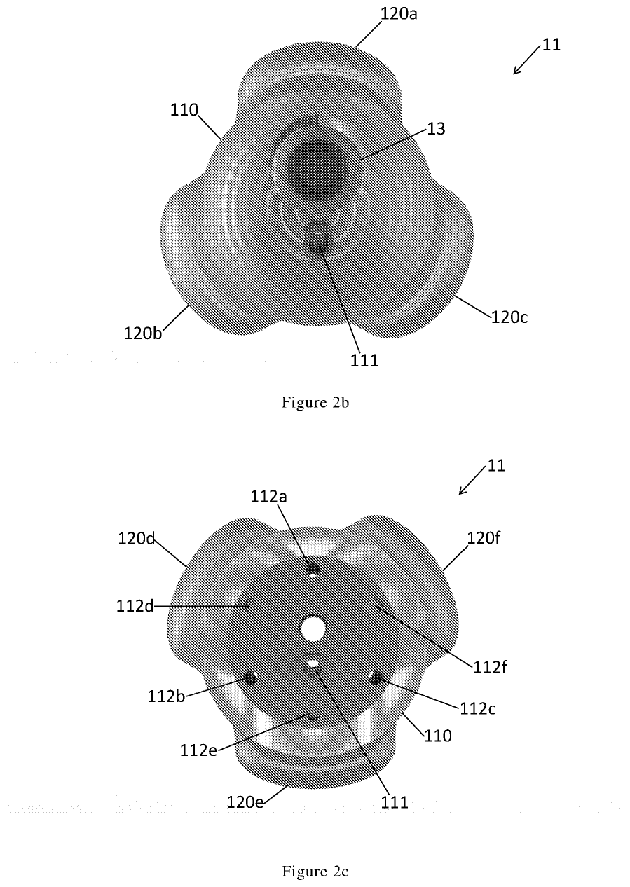

FIGS. 2a to 2c each show a schematic view of a positioning device 11 of a system according to an embodiment of the invention. FIG. 2a shows an isometric view, FIG. 2b shows a front view and FIG. 2c shows a rear view. In this embodiment, the system is for positioning a needle 15 within an ear canal of a patient. In particular, the system is for aligning the needle 15 with the centre of the posteroinferior quadrant of the tympanic membrane for the purpose of piercing the tympanic membrane at this location to deliver medication to perfuse via the round window of the cochlea. It will be appreciated that this embodiment is merely illustrative and that the invention extends to positioning other tools within other lumens.

In the embodiment of FIGS. 2a to 2c, the cylindrical body 110 comprises a core and a membrane surrounding the outer surface of the core. The positioning device 11 comprises six actuators 120a-f each configured to apply a radial force to an internal wall of the ear canal to move the cylindrical body 110 in a different radial direction. In this embodiment the actuators 120a-f take the form of inflatable chambers. The cylindrical body 110 comprises six inflation channels 112a-f, each inflation channel 112a-f being configured to supply an inflation fluid to one of the actuators 120a-f. In this embodiment, the inflation fluid is deionised water, but in other embodiments the inflation fluid may be another suitable fluid, not necessarily limited to a liquid. Each actuator 120a-f comprises a portion of the membrane which is inflatable to provide a radial force to an internal wall of an ear canal to move the cylindrical body 110. In other embodiments, alternative actuators may be used.

Both the core and the membrane of the cylindrical body are formed from a compliant material, such as silicone-based rubber. The core comprises a stiffness which is greater than a stiffness of the membrane; this means that the radial force resulting from inflation of a portion of the membrane forming part of an actuator 120a-f causes the cylindrical body 110 to move, rather than causing the core to deform without radial movement of the cylindrical body 110 as a whole.

The cylindrical body 110, without any of the actuators 120a-f being inflated, has a diameter of approximately 4 mm and a length of approximately 10 mm. The thickness of the membrane is approximately 1 mm. These dimensions are suitable for the application of the positioning device 11 to a human ear canal, which is, on average, 7 mm in diameter and 25 mm in length. It will be appreciated that the dimensions of the positioning device 11 will vary depending on the particular application of the positioning device 11.

The actuators 120a-f are arranged in two stages spaced apart along a longitudinal axis ‘A’ of the cylindrical body 110. Each stage comprises three actuators. The actuators within each stage are spaced apart equidistantly about the longitudinal axis ‘A’. The stages are radially offset from one another by 45 degrees. The arrangement of the six actuators 120a-f enables the longitudinal axis ‘A’ of the cylindrical body 110 to be moved in four degrees of freedom when the positioning device 11 is arranged within an ear canal. The longitudinal axis ‘A’ of the cylindrical body can be translated horizontally and vertically relative to a longitudinal axis of the ear canal. The cylindrical body 110 can also be tilted horizontally and vertically about the centre ‘C’ of the cylindrical body, i.e. the cylindrical body can be rotated about the ‘x’ and ‘y’ axes centred at the centre ‘C’ of the cylindrical body as shown in FIG. 2a, to change the angular orientation of the longitudinal axis ‘A’ of cylindrical body 110 relative to a longitudinal axis of the ear canal. Each of the actuators 120a-f can be controlled independently so that one or more of the actuators 120a-f can be inflated at the same time to move the cylindrical body 110.

The Jacobian matrix of the cylindrical body 110 can be obtained from the wrenches induced by the actuators 120a-f. In use, a single actuator ‘i’ will induce a radial force ‘f_{act,i}’ on the cylindrical body 110. This force induces a wrench ‘ω_{ctr}’ at the centre ‘C’ of the cylindrical body 110, such that:

$${w}_{ctr} = {Ad}_{act , i} \cdot {w}_{act , i}$$

where ‘ω_{act,i}’ is the wrench induced on the cylindrical body 110 local to the actuator ‘i’, equal to the ω_{act,i}=[0, ∫_{act,i}, 0, 0.0]^{T}, and ‘Ad_{act,i}’ is the wrench transformation matrix of actuator ‘i’. The wrench transformation matrix can be obtained assuming a rigid transformation of the cylindrical body 110 at the actuator ‘i’ relative to the centre ‘C’ of the cylindrical body 110. The actuation matrix of the cylindrical body 110 ‘H’ can be defined as:

$$H = [ {Ad}_{2 , 1} , … , {Ad}_{2 , n} ]$$

where ‘Ad_{2,i}’ is the second column of the wrench transformation matrix of actuator ‘i’, the inverse Jacobian matrix of the cylindrical body 110 is then defined, relative to the actuation matrix of the cylindrical body 110, as:

$${J}^{- 1} = {H}^{T}$$

The inverse Jacobian matrix can be defined in terms of a 6×4 matrix as:

$${J}^{- 1} = [ \begin{matrix}0 & - 1 & b & 0 \\ \frac{\sqrt{3}}{2} & \frac{1}{2} & - \frac{1}{2} b & \frac{\sqrt{3}}{2} b \\ - \frac{\sqrt{3}}{2} & \frac{1}{2} & - \frac{1}{2} b & - \frac{\sqrt{3}}{2} b \\ 0 & 1 & b & 0 \\ - \frac{\sqrt{3}}{2} & - \frac{1}{2} & - \frac{1}{2} b & \frac{\sqrt{3}}{2} b \\ \frac{\sqrt{3}}{2} & - \frac{1}{2} & - \frac{1}{2} b & - \frac{\sqrt{3}}{2} b\end{matrix} ]$$

where ‘b’ is the distance between the actuator ‘i’ and the centre ‘C’ of the cylindrical body 110. The inverse Jacobian matrix can then be used to solve the inverse differential kinematics of the cylindrical body 110 as:

$$δ q = {J}^{- 1} \cdot δ X$$

where δX=[δₓ, δ_{y}, θₓ, θ_{y}]\hat{ }^{T} is the relative pose change of the cylindrical body 110 in the frame of reference of the tip. “δₓ” and ‘δ_{y}’ represent translational changes and ‘θₓ’ and ‘θ_{y}’ represent rotational changes. ‘δ_{q}’ is the vector of radial extensions of the actuators when inflated. The above approach accounts for directional decomposition of the actuator motion, but does not account for compliance of the actuator ‘i’ or changes in the configuration of the actuator ‘i’ under expansion.

The cylindrical body 110 of the embodiment of FIGS. 2a to 2c comprises a longitudinal bore 111 for receiving the needle 15. In use, the needle 15 extends through the longitudinal bore 111 and extends out from the front of the positioning device 11. The longitudinal axis of the longitudinal bore 111 lies along the longitudinal axis ‘A’ of the cylindrical body 110 such that moving the longitudinal axis ‘A’ of the cylindrical body 110 to a target location and orientation has the effect of moving the longitudinal axis of the longitudinal bore 111 to the target location and orientation. When the needle 15 is then inserted into the longitudinal bore 111, the longitudinal axis of the needle 15 therefore adopts the target location and orientation. The system of this embodiment further comprises a camera 13 arranged at the front of the positioning device 11. In other embodiments, the camera 13 may be provided separately from the positioning device 11. The function of the camera 13 is described in further detail below. In other embodiments, the system may comprise optical fibres in place of the camera 13. In such embodiments, the optical fibres provide the same function as the camera 13 as described below.

The diameter of the longitudinal bore 111 and the diameter of the needle 15 are such that a clearance fit is provided between the needle 15 and the longitudinal bore 111. This allows the needle 15 to move, in the longitudinal direction, within the longitudinal bore 111 relative to the cylindrical body 110, while limiting radial movement of the needle 15 within the longitudinal bore 111.

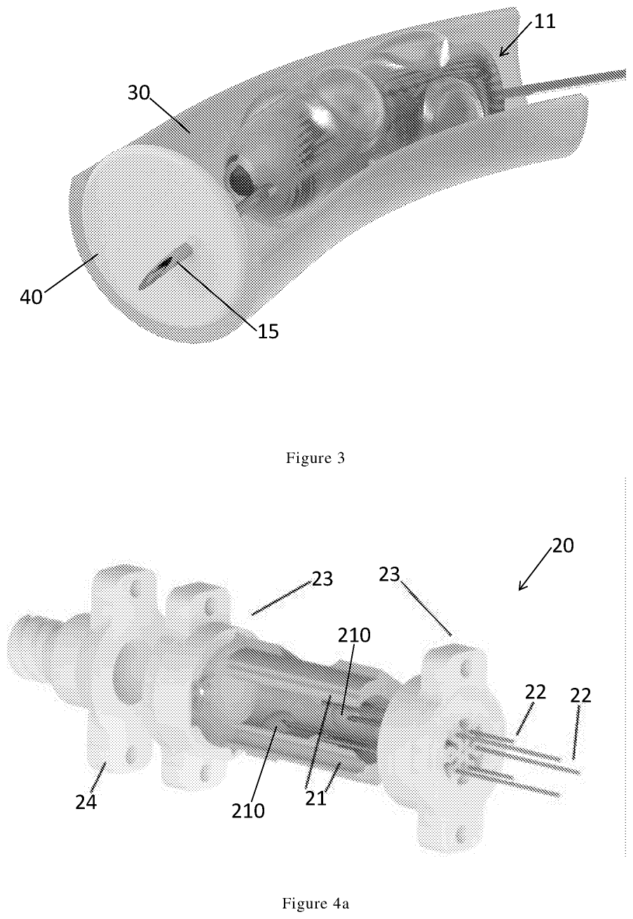

FIG. 3 shows a schematic representation of the positioning device 11 of FIGS. 2a to 2c in use within an ear canal. The needle 15 is shown as piercing the tympanic membrane 40, for example for the purpose of administering an intratympanic injection.

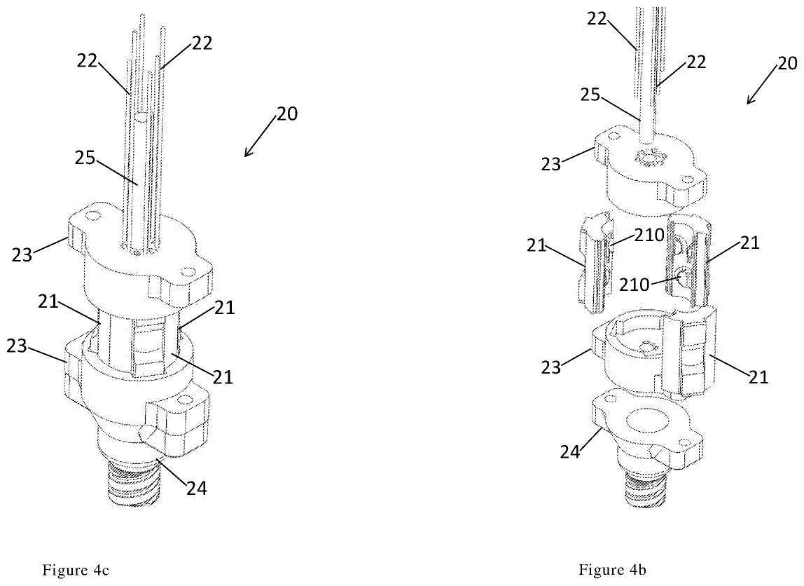

FIGS. 4a-c show a schematic representation of a moulding apparatus 20 for producing the positioning device 11 of FIGS. 2a to 2c. The apparatus 20 comprises multiple shell elements 21 which together form a shell defining an internal moulding volume. The shell is open at both ends. The internal surface of the shell comprises nodules 210 corresponding to the location of the actuators 120a-f of the positioning device 11. The apparatus 20 comprises an endcap 23 arranged at either end of the shell, and a funnel 24 arranged in fluid communication with the internal moulding volume. The funnel 24 is attachable to the endcap 23 at the end of the shell corresponding to the front of the positioning device 11.

Extending through the other end cap 23, at the end of the shell corresponding to the rear of the positioning device 11, and into the internal moulding volume are six wires 22 (one of the wires is omitted from FIG. 4 for clarity). In this example, the wires 22 are formed of steel of approximately 0.5 mm diameter. The wires 22 are arranged so as to form the inflation channels 112a-f of the positioning device 11 during a moulding process. The apparatus 20 further comprises a round bar 25 (as shown in FIGS. 4b and 4c). The end cap 23 at the end of the shell corresponding to the rear of the positioning device 11 is configured to receive the round bar 25, such that the round bar 25 extends through the end cap 23 into the internal moulding volume.

In a first stage of producing the positioning device 11 of FIGS. 2a to 2c, the core of the cylindrical body 110 is produced. Liquid material, such as liquid rubber, is introduced into the internal moulding volume via the funnel 24. The liquid material fills the internal moulding volume to form the core, with the nodules 210 creating corresponding cavities in the external surface of the core, the wires 22 creating the inflation channels 112a-f and the round bar 25 creating a channel to receive the camera 13, or optical fibres in other embodiments. FIG. 4c shows the assembled moulding apparatus 20 after insertion of the wires 22 and round bar 25. The length of the wires 22 and round bar 25 are selected such that they protrude out of the internal moulding volume to facilitate easy removal of the wires 22 and round bar 25 after the core of the cylindrical body 110 has been formed.

The longitudinal bore 111 of the cylindrical body 110 may be produced in a similar manner to the cavities, the inflation channels 112 and the channel to receive the camera 13 or optical fibres. Once the liquid material has set, the endcaps 23 and the wires 22 and round bar 25 are removed, the shell elements 21 are separated and the finished core of the positioning device 11 is removed.

It will be appreciated that this method is particularly suited for producing a positioning device for intratympanic procedures, for example a positioning device comprising a cylindrical body with a maximum diameter in the range of 3-5 mm and a maximum length in the ranged of 6-14 mm. The dimensions of the positioning device can be scaled to fit the desired use-case as well as to adapt to the patient anatomy. This can be achieved by altering the radius and the height of the shell elements 21 of the apparatus.

It will be appreciated that the minimum size of the positioning device may be subject to a number of constraints. Reducing the diameter is possible while sufficient clearance between the actuators and the camera is maintained. Altering the size of the camera or imaging device thus contributes to scaling the diameter of the device. The minimum length of the positioning device is primarily defined by the size of the actuators. The actuators can be scaled to allow for altering the movement range of the positioning device, where a larger diameter enables greater radial deformation. The length of the camera or other imaging device may be short compared to the total length of the positioning device. A decrease in stiffness of the positioning device when the actuators are inflated can be overcome by simply replacing the membrane material with a harder rubber.

After the first stage of producing the positioning device 11, the core is over-moulded with the membrane. Before the membrane is applied to the core, the cavities in the external surface of the core are filled with a gel wax or other suitable material such that the gel wax lies flush with the external surface of the core. This prevents the material used to form the membrane from filling the cavities during the over-moulding process. The core is then over-moulded to form the membrane by a suitable process such as injection moulding. For example, the core may be placed in a mould comprising shell elements defining an internal moulding volume which is larger than the internal moulding volume defined by the shell elements 21 of the moulding apparatus 20 and which does not comprise nodules. Liquid membrane material may then be injected into the mould to form the membrane. The membrane may be formed from silicone rubber. Once the membrane is fully cured the gel wax is melted and removed via the inflation channels 112a-f. Each actuator 120a-f of the positioning device 11 therefore comprises a cavity in the external surface of the core of the cylindrical body 110 and a portion of the membrane overlaying the cavity. Each inflation channel 112a-f is in fluid communication with one of the cavities so as to supply an inflation fluid to the respective actuator 120a-f. Supplying pressurised inflation fluid to an actuator 120a-f causes the respective cavity to fill, which causes the respective overlaying portion of the membrane to expand. This causes the actuator 120a-f to apply a radial force to an internal wall of an ear canal in use.

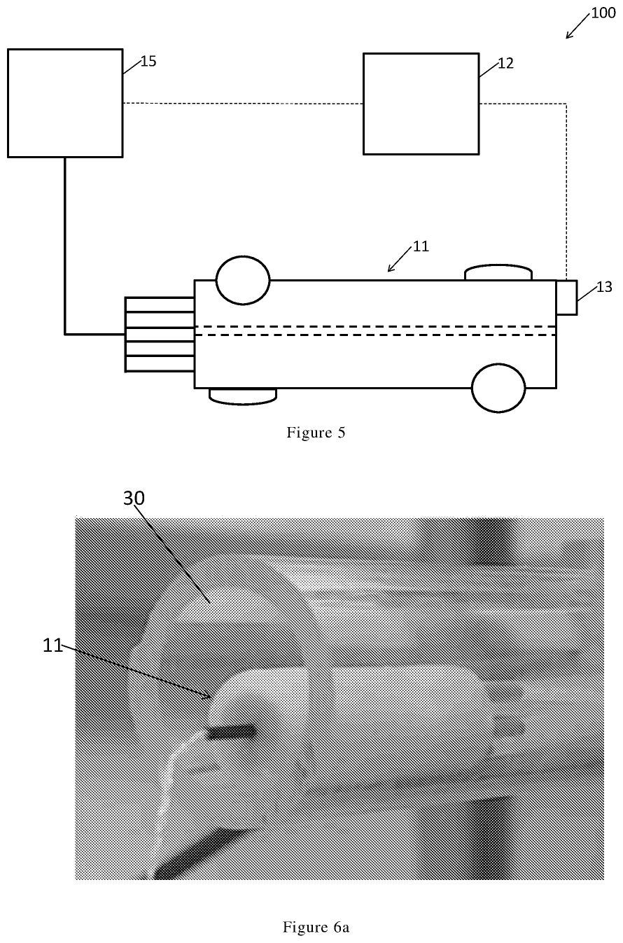

FIG. 5 shows a system 100 according to an embodiment of the invention. The system 100 of FIG. 5 shares features in common with the system 10 of FIG. 1 and like reference numerals are used to refer to like features. The system 100 of FIG. 5 comprises the positioning device 11 of FIGS. 2a to 2c. For clarity, not all the features of the positioning device 11 are shown in FIG. 5. In addition to the positioning device 11, the camera 13 and the control system 12, the system comprises a positive displacement pump 15. In some embodiments, the positive displacement pump 15 may comprise a piston received within a cylinder, e.g. a syringe pump. In other embodiments, any other suitable pump may be used in place of the positive displacement pump. In this embodiment, the pump 15 is electronically actuated. The dashed lines extending from the control system 12 to the pump 15 and the camera 13 indicate that the control system 12 is in communication with the pump 15 and the camera 13 so as to send and/or receive signals from each of the pump 15 and the camera 13. The solid lines extending from the pump 15 to the positioning device 11 indicate that the pump 15 is in fluid communication with each of the inflation channels of the cylindrical body via a manifold, so as to supply inflation fluid to each of the actuators. The system 100 is operable so as to select which of the actuators to deliver inflation fluid to, so as to independently operate each of the actuators. This may be achieved through the use of electronic valves controlled by the control system.

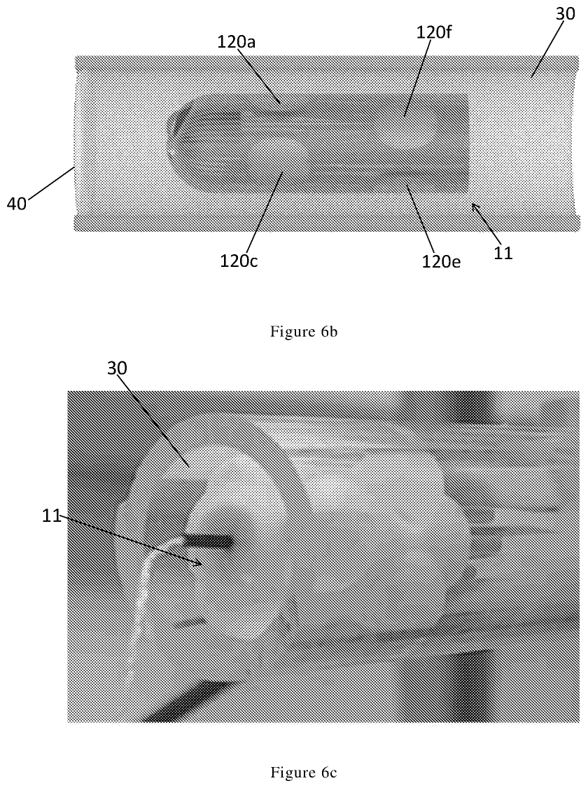

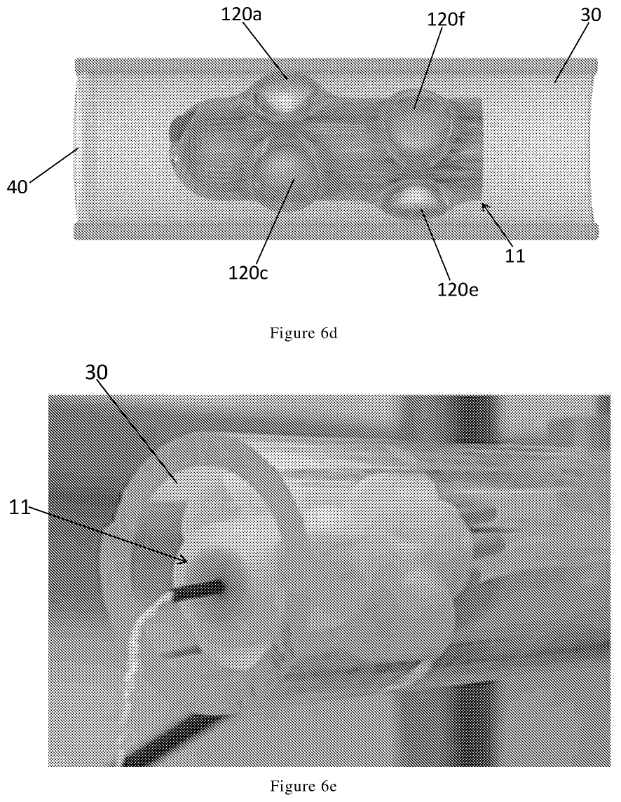

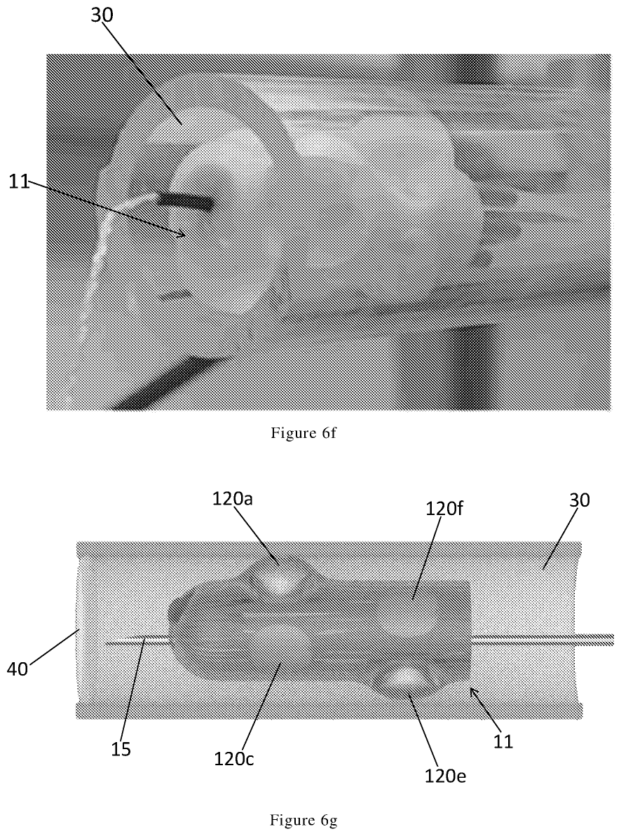

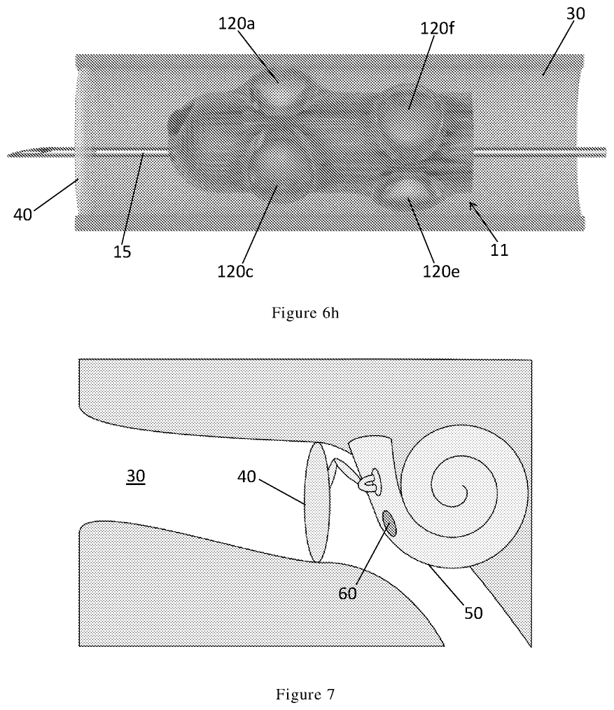

FIGS. 6a to 6h illustrate schematically how the positioning device 11 of FIGS. 2a to 2c can be maneuvered within an ear canal 30 in one illustrative example. In FIGS. 6a, 6c, 6e and 6f, the ear canal 30 is represented by a transparent tube in an experimental set-up. In use, a user of the system, such as a surgeon or other medical professional, will begin by inserting the positioning device 11 into the ear canal 30 as shown in FIGS. 6a and 6b. All of the actuators 120a-f are then inflated so as to stabilise the positioning device 11 within the ear canal 30, as shown in FIGS. 6c and 6d. This has the effect of retaining the longitudinal position of the positioning device 11 within the ear canal 30. The actuators 120a-f are then independently inflated to move the cylindrical body 110 to adopt a target position, as shown in FIGS. 6f and 6g (in the example of FIG. 6g, actuators 120a and 120e are inflated, and actuators 120c and 120f are not inflated). FIGS. 6e and 6f illustrate different target positions that can be achieved with the actuators 120a-f.

Once the cylindrical body 110 has adopted the target position, e.g. once the longitudinal bore 111 of the cylindrical body 110 has been aligned with the centre of the posteroinferior quadrant of the tympanic membrane, the user inserts the needle 15 through the longitudinal bore 111 as shown in FIG. 6g. The needle 15 may then be moved, in the longitudinal direction within the longitudinal bore 111 and relative to the cylindrical body 110, to pierce the tympanic membrane 40 as shown in FIG. 6h. FIG. 6h also shows further operation of the actuators 120a-f, for example in response to an unintended movement of the needle 15 by the user, to maintain the position of the cylindrical body 110. The position of the cylindrical body 110 at the target position is maintained throughout the process of inserting the needle 15 through the longitudinal bore 111 and piercing the tympanic membrane 40.

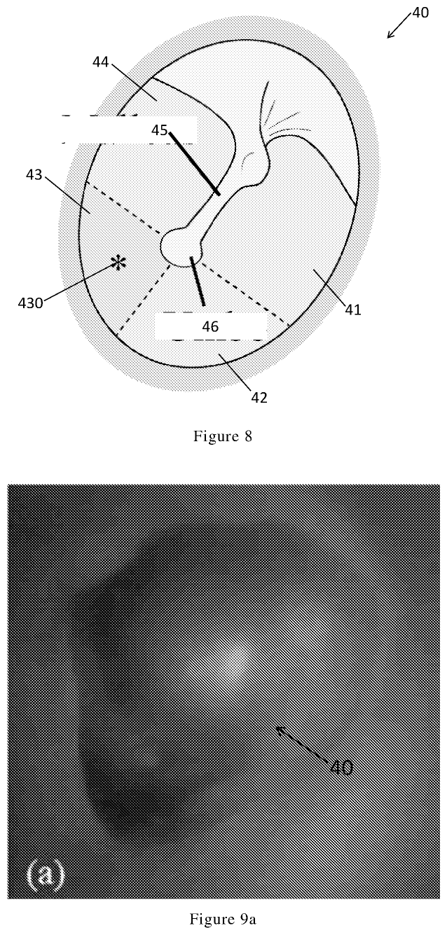

FIG. 7 shows a schematic representation of an ear, showing the ear canal 30, the tympanic membrane 40, the cochlea 50, and the round window 60 of the cochlea 50. FIG. 8 shows a schematic representation of the tympanic membrane 40, showing the anterosuperior quadrant 41, the anteroinferior quadrant 42, the posteroinferior quadrant 43, the centre of posteroinferior quadrant 430, the posterosuperior quadrant 43, the malleus 45 and the umbo 46.

The system 10, 100 of FIG. 1 or FIG. 5, comprising the positioning device 11 of FIGS. 2a to 2c, is configured to autonomously move the cylindrical body 110 to adopt a target position. The control system 12 is configured to automatically obtain an input signal indicative of the target position and automatically provide an output signal to control the actuators 120a-f to move the cylindrical body 110 to adopt the target position. This is achieved through processing an image captured by the camera 13 to identify a target location in the image and moving the cylindrical body 110 using the actuators accordingly 120a-f. In other embodiments, the system 10, 100 may not be configured to autonomously move the cylindrical body 110 to adopt a target position. For example, a human user of the system 10, 100 may manually provide an input signal to the control system 12 to move the cylindrical body 110 to the target position.

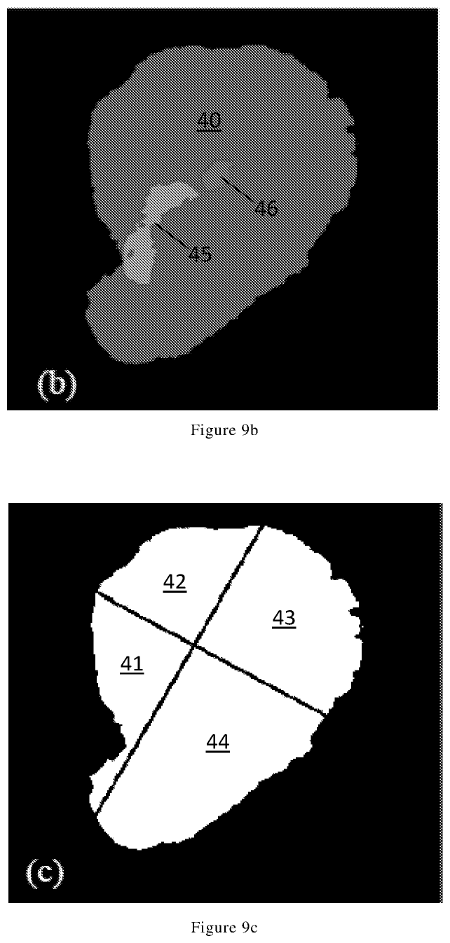

FIGS. 9a to 9d illustrate a process by which the control system 12 of the system 10, 100 of FIG. 1 or FIG. 5 processes an image captured by the camera 13 to produce an input signal indicative of a target position for the cylindrical body 110. FIG. 9a shows an actual image captured by the camera 13 in which the tympanic membrane 40 is visible. FIG. 9b shows the image of FIG. 9a processed by the control system 12 to identify the outline of the tympanic membrane 40, the umbo 46 and the malleus 45. In this embodiment, the control system 12 utilises a deep artificial neural network trained to identify the outline of the tympanic membrane 40, the umbo 46 and the malleus 45, as well as the background of the image.

The neural network performs semantic segmentation to label each of the pixels in the image, i.e. label each pixel as representing the umbo 46, the malleus 45 or the remainder of the tympanic membrane 40. The neural network may comprise a spatial pyramid pooling module and an encoder-decoder structure. An example of such a neural network is DeepLabV3+(L.-C. Chen, Y. Zhu, G. Papandreou, F. Schroff, and H. Adam, “Encoder-decoder with atrous separable convolution for semantic image segmentation,” in Proceedings of the European conference on computer vision (ECCV), pp. 801-818, 2018). In other embodiments, a different type of neural network or a different method of image processing may be used. In this embodiment, the neural network is implemented on a mobile architecture such as MobileNetV2 (M. Sandler, A. Howard, M. Zhu, A. Zhmoginov, and L. C. Chen, “MobileNetV2: Inverted Residuals and Linear Bottlenecks,” Proceedings of the IEEE Computer Society Conference on Computer Vision and Pattern Recognition, pp. 4510-4520, 2018). In other embodiments, any other suitable architecture may be used.

In an example implementation, the neural network was trained using both actual images of human tympanic membranes and images of a phantom tympanic membrane of a medical training device. From a set of 277 images of human tympanic membranes of 500×500 resolution and 80 phantom images of 640×480 pixels, 216 human images and 60 phantom images were used for training and the remaining images were used for testing. Each pixel in each of the training images was labelled as representing the umbo, the malleus, the remainder of the tympanic membrane or the background of the image, i.e. any pixels of the image which do not represent the umbo, the malleus or the remainder of the tympanic membrane.

In an experimental set-up to test the neural network, the camera used was of a different resolution to the images used to train the neural network. The experimental camera had a resolution of 200×200 with a heavy RGB colour shift. As a result, it was expected that the trained network may produce inaccurate results due to training data distribution being significantly different from the test data distribution. To overcome this issue, data augmentation was applied including applying scale, rotation and perspective transforms, horizontal and vertical flips, RGB colour shifts, brightness, contrast, saturation and blurring operations at random with a probability of 0.5 during the training process.

An Adam optimiser was used with a learning rate of 0.001. The neural network was trained for 300 epochs with early stopping. Intersection over Union (IoU) was recorded. IoU is a standard performance metric for segmentation evaluation that quantifies the overlap between ground-truth labels, i.e. labels manually applied to images used for training, and predicted labels, i.e. labels obtained using a trained neural network. An IoU of 0.80 was achieved on the test set, which was comprised of the remaining 61 human images and 20 phantom images. A value close to 1 suggests high overlap.

After the image is processed to identify the outline of the tympanic membrane 40, the umbo 46 and the malleus 45, the image is further processed to identify each of the anterosuperior 41, anteroinferior 42, posteroinferior 43 and posterosuperior 44 quadrants, as shown in FIG. 9c. The umbo 46 and the malleus 45 are used as references to identify the quadrants. A first axis runs longitudinally along the centre of the malleus 45, separating the antero 41, 42 and postero 43, 44 quadrants, and a second axis runs perpendicularly to the first axis, intersecting the centre of the umbo 46 and separating the inferior 42, 43 and superior 41, 44 quadrants. Once the posteroinferior quadrant 43 has been identified, the centre 430 of posteroinferior quadrant can be located. FIG. 9d shows the posteroinferior quadrant 43 and its centre 430 overlaid on the original image captured by the camera 13. The centre 430 of the posteroinferior quadrant 43 can then be used to produce the input signal to move the cylindrical body 110 to the target position, i.e. to align the longitudinal axis of the longitudinal bore 111, and thus the needle 15, with the centre 430 of the posteroinferior quadrant 43.

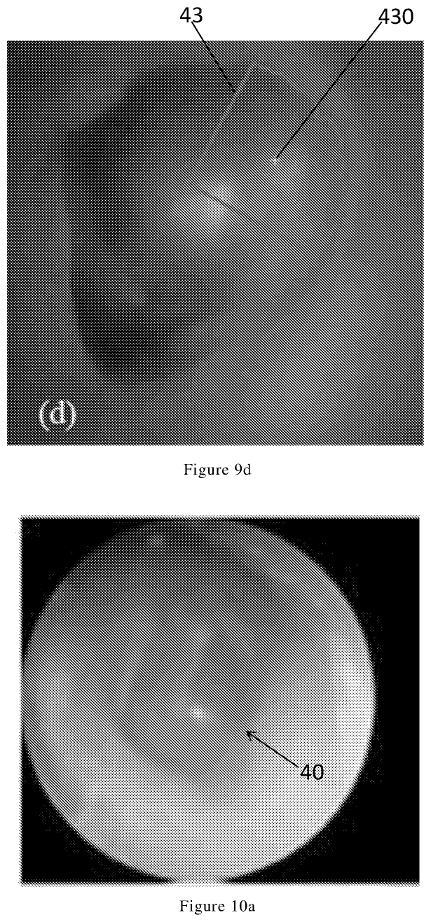

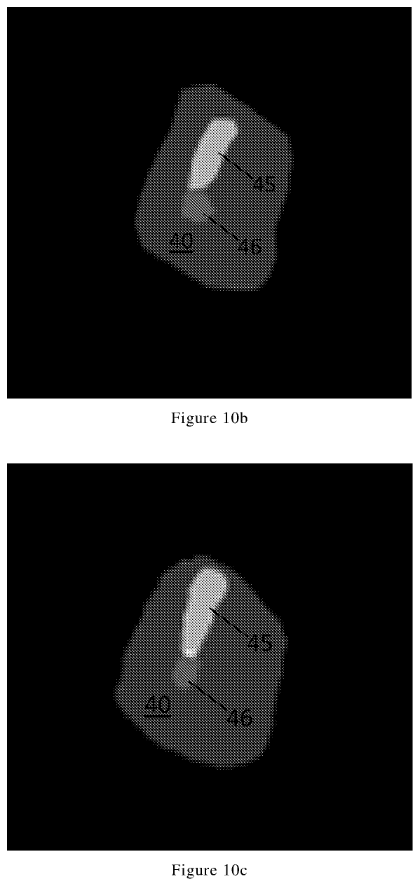

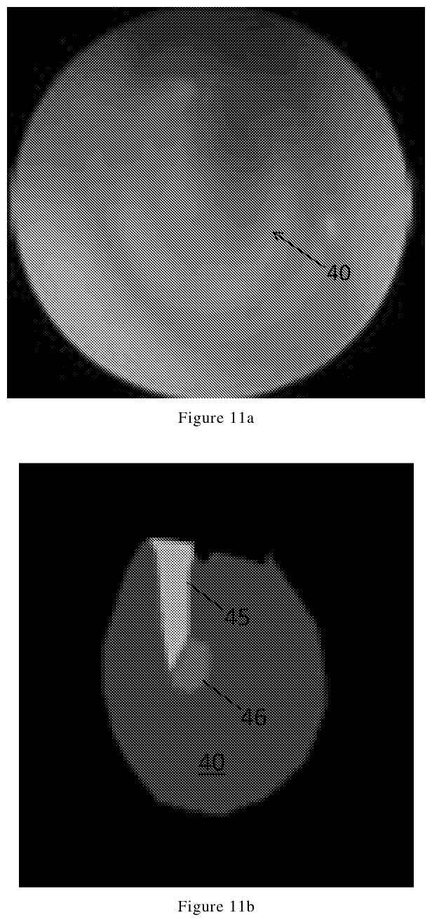

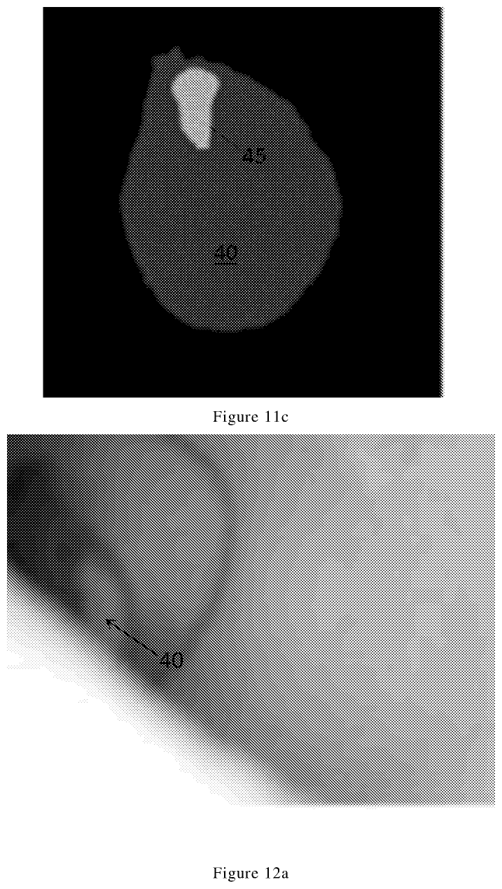

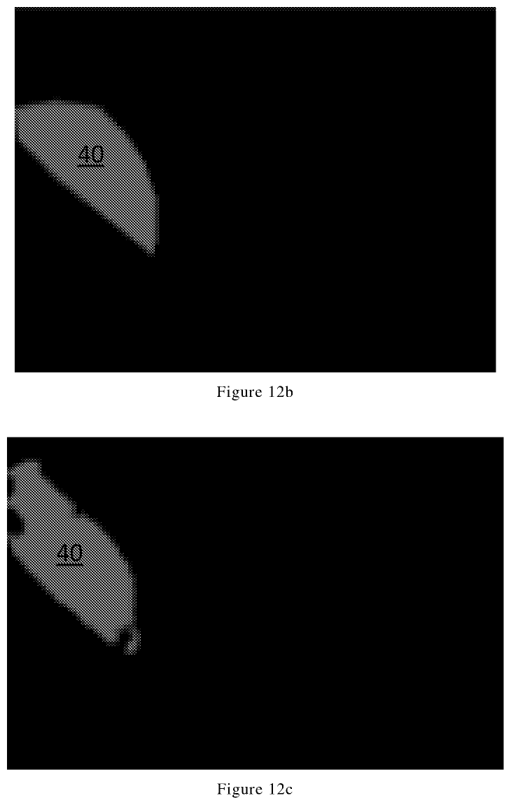

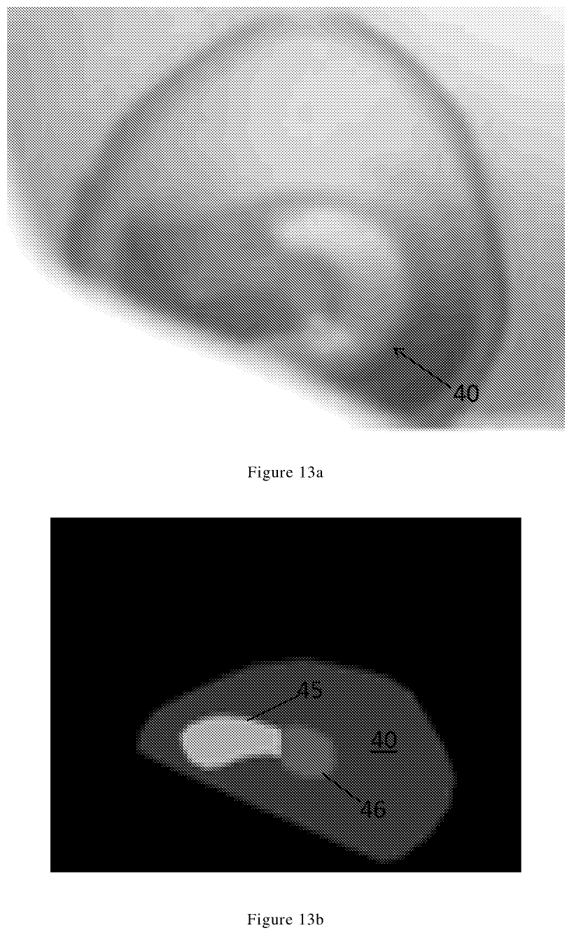

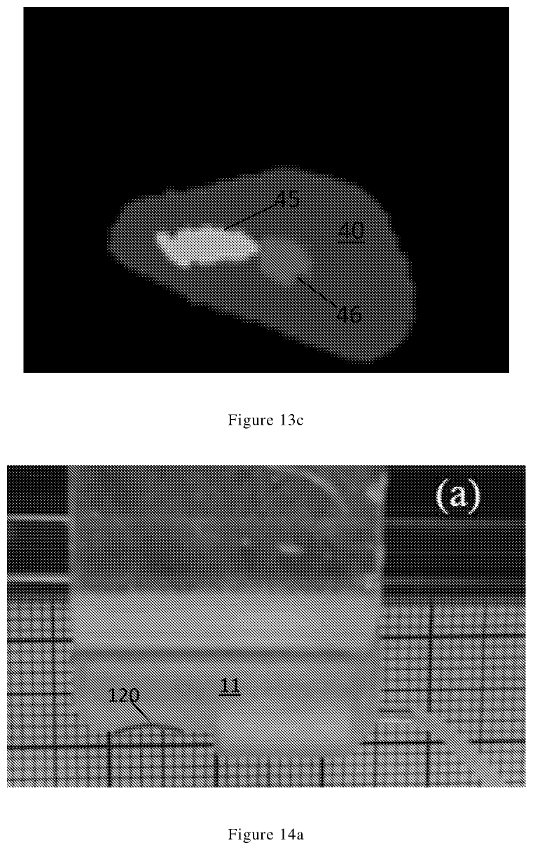

FIGS. 10a to 10c and 11a to 11c show some results of processing of images of human tympanic membranes 40 using the neural network. FIGS. 10a and 11a each show an original image of a human tympanic membrane 40. In FIG. 10a, the umbo 46 is visible and in FIG. 11a the umbo 46 is not visible. FIGS. 10b and 11b show labelled training images obtained from the respective original image. FIGS. 10c and 11c show images labelled using the neural network to process the respective original image. As shown in FIG. 11c, the umbo 46 has not been identified by the neural network because it is not visible in the original image. FIGS. 12a to 12c and 13a to 13c show some results of processing of images of phantom tympanic membranes 40 using the neural network. FIG. 12a shows an original image of a phantom tympanic membrane 40 in which the umbo 46 and malleus 45 are not visible, and the tympanic membrane 40 itself is only partially visible. FIG. 13a shows an original image of a phantom tympanic membrane 40 in which the umbo 46, the malleus 45 and the tympanic membrane 40 itself are partially visible. FIGS. 12b and 13b show labelled training images obtained from the respective original image, and FIGS. 12c and 13c show images labelled using the neural network to process the respective original image.

Once the input signal has been produced, the control system 12 then automatically determines an output signal to control the actuators 120a-f to move the cylindrical body 110 to adopt the target position. In this embodiment, the output signal comprises a volume of inflation fluid to be sent to each actuator 120a-f to provide the required radial movement. The relationship between inflation volume and radial displacement of each actuator 120a-f is predetermined experimentally and the control system 12 stores a look-up table to determine the volume of inflation fluid to deliver to each actuator 120a-f to provide the required displacement. In other embodiments, different control values, for example inflation pressure, may be used to provide the required movement of the cylindrical body 110.

The system 10, 110 is further configured to compensate for undesired movement of the needle 15 in use, for example due to an unsteady hand of a user of the system 10, 110. The control system 12 is configured to receive a further input signal indicative of a deviation of the cylindrical body 110 from the target position. Undesired motion may be inferred from changes in images captured by the camera, for example using optical flow-base analysis, or from monitoring changes in the pressure of inflation fluid within one or more of the actuators 120. In some embodiments, both of these methods may be employed in combination to provide the accuracy advantages of the image-based method with the fast response of the pressure-based method. The system 10, 110 then automatically determines a further output signal to control the actuators 120a-f to move the cylindrical body 110 back to the target position.

During the development of the invention, a number of experimental validations were performed. To test the mechanical performance of the positioning device 11, three prototypes were produced each having a membrane made from a different platinum-catalysed silicone rubber. The three rubbers used were Ecoflex® 00-30 (EF30), Ecoflex® 00-50 (EF50) and Dragon Skin Fx Pro® (DSFX) from Smooth-On Inc., Pennsylvania, USA. It will be appreciated that these are just example test materials and are not limitations on the materials used to produce the positioning device 11. During testing, inflation fluid was provided to the inflation channels 112a-f of the prototypes using platinum-cured silicone rubber tubing having an inner diameter of 0.5 mm and an outer diameter of 1 mm. The inflation fluid was pumped to the actuators 120a-f using a stepper motor-driven syringe pump with a 3 ml syringe controlled by a six-axis motion controller.

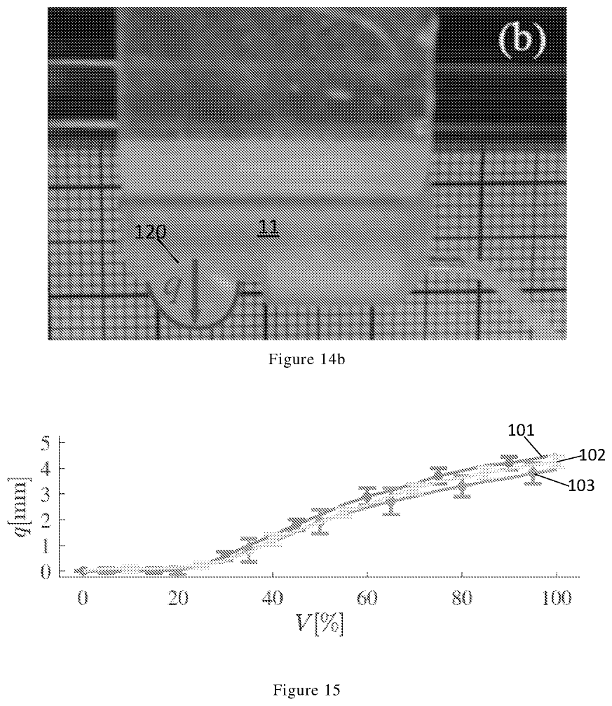

FIGS. 14a and 14b show an experimental set-up used to measure the radial displacement of an actuator 120 when supplied with a varying volume of inflation fluid. Each actuator of each of the prototypes discussed above was tested. FIG. 14a shows the actuator 120 in a non-inflated state and FIG. 14b shows the actuator 120 supplied with inflation fluid and displaced by a distance ‘q’. The distance ‘q’ was measured as the furthest distance from the baseline of the actuator 120, i.e. the position of the actuator 120 when not inflated. FIG. 15 shows a plot of mean actuator displacement ‘q’ in mm and standard deviation against percentage of a maximum volume ‘V’ of supplied inflation fluid for each of the three prototype membrane materials: EF30 (line 101), EF50 (line 103) and DSFX (line 102). The maximum volume was the maximum volume achievable before failure of the actuator 120, found experimentally. In this example the maximum volume was 0.57 ml.

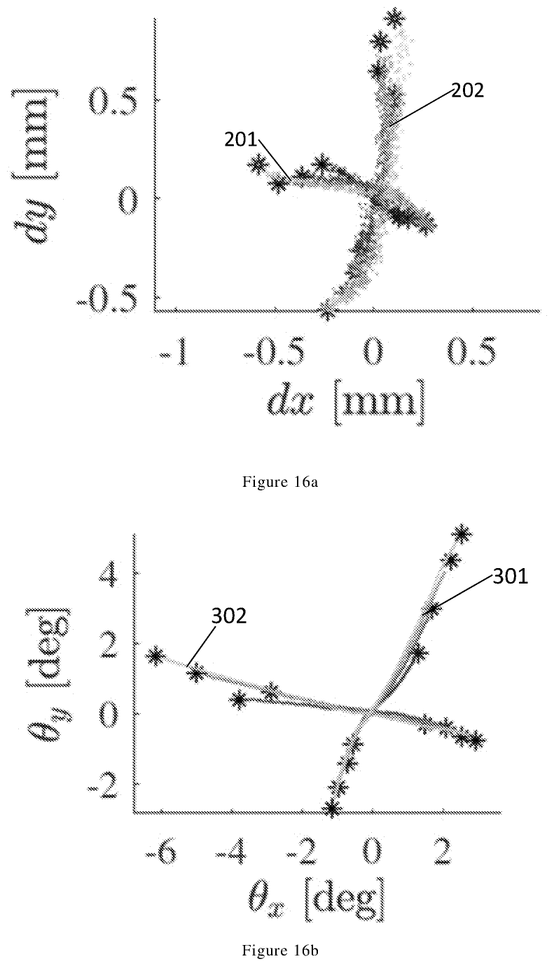

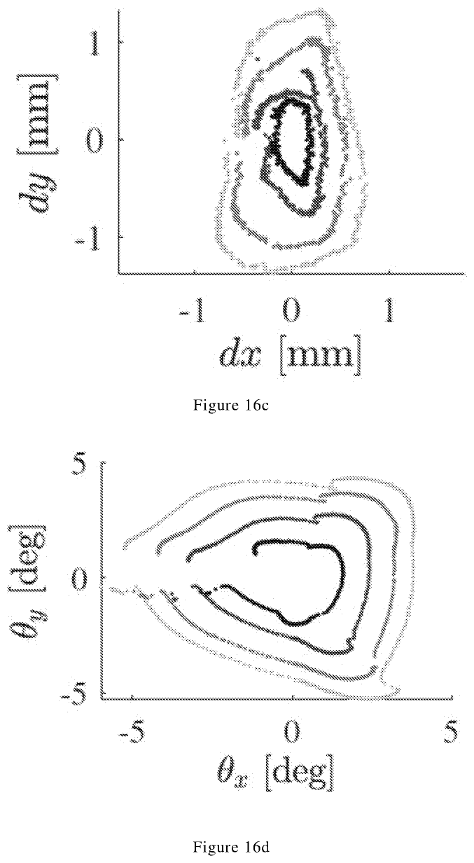

In order to test the ability of the actuators to accurately move the cylindrical body to a target position, a prototype positioning device was placed inside an acrylic tube of 14 mm internal diameter, representing a scaled-up ear canal, and the actuators were controlled according to a number of commands. Initially, all of the actuators were simultaneously actuated in order to align the longitudinal axis of the longitudinal bore of the cylindrical body with the longitudinal axis of the acrylic tube. An electromagnetic tracker, representing a needle, was placed within the longitudinal bore to track the movement of the cylindrical body. FIG. 16a shows the tracked linear translation of the centre of the cylindrical body in the horizontal 201 and vertical 202 directions (corresponding to the axes centred at ‘C’ in FIG. 2a).

Four trajectories in the horizontal and vertical directions were performed, ranging from a negative maximum displacement from the origin to a positive maximum displacement from the origin. The maximum displacements are defined as: 1 mm, 1.5 mm, 2 mm and 2.5 mm. Each asterisk in FIG. 16a represents the start or end point of one of the four trajectories. The individual points in FIG. 16a represent positions recorded by the electromagnetic tracker as the cylindrical body moved along a trajectory. To allow for easier comparison, each trajectory shown in FIG. 16a has been offset by the mean location of the recorded positions to centre the trajectory at ‘0, 0’. The deviations from the intended trajectories evident in FIG. 16a may have been the result of uncertainties in the amount of force applied by the actuators to the internal wall of the acrylic tube to centre the cylindrical body within the acrylic tube prior to performing the intended trajectory. In some embodiments of the invention, these uncertainties may be addressed by monitoring the pressure of inflation fluid within one of more of the actuators to determine a contact force between the actuator(s) and the internal wall of a lumen. These contact forces may be incorporated in a model, used to control the movement of the cylindrical body, which takes into account any elastic behaviour of the positioning device.

FIG. 16b shows the tracked rotation, i.e. the tracked tilting of the longitudinal axis of the longitudinal bore of the cylindrical body, about the ‘x’ and ‘y’ axes (lines 302 and 301 respectively) extending from the centre ‘C’ of the cylindrical body (see FIG. 2a). Four tilting trajectories about the ‘x’ and ‘y’ axes extending from the centre ‘C’ of the cylindrical body were performed, ranging from a negative maximum tilt from the origin to a positive maximum tilt with respect to the original frame ‘C’. The maximum tilts are defined as: 7.5 degrees, 10 degrees, 12.5 degrees and 15 degrees. As in the case of FIG. 16a, individual points in FIG. 16b represent positions recorded by the electromagnetic tracker as the cylindrical body moved along a trajectory, and the trajectories have been centred about ‘0, 0’.

FIGS. 16a and 16b show the results of controlling translation and tilting of the cylindrical body to produce linear trajectories of the longitudinal axis of the longitudinal bore of the cylindrical body. Translation and tilting of the cylindrical body were also controlled to produce circular trajectories of the longitudinal axis of the longitudinal bore of the cylindrical body. FIG. 16c shows tracked circular trajectories of the longitudinal axis of the longitudinal bore of the cylindrical body when the cylindrical body is controlled to perform a circular motion in translation, i.e. with the longitudinal axis of the longitudinal bore of the cylindrical body remaining parallel to the longitudinal axis of the acrylic tube. FIG. 16d shows tracked circular trajectories of the longitudinal axis of the longitudinal bore of the cylindrical body when the cylindrical body is controlled to perform a circular motion in rotation, i.e. with the longitudinal axis of the longitudinal bore of the cylindrical body being tilted with respect to the longitudinal axis of the acrylic tube. Four circular trajectories, with radii of 1 mm, 1.5 mm, 2 mm and 2.5 mm respectively, were produced by moving the cylindrical body in translation, and four circular trajectories were produced by moving the cylindrical body in rotation, at tilt angles with respect to the longitudinal axis of the acrylic tube of 7.5 degrees, 10 degrees, 12.5 degrees and 15 degrees respectively. The positions recorded by the electromagnetic tracker as the circular trajectories were performed are plotted in FIGS. 16c and 16d.

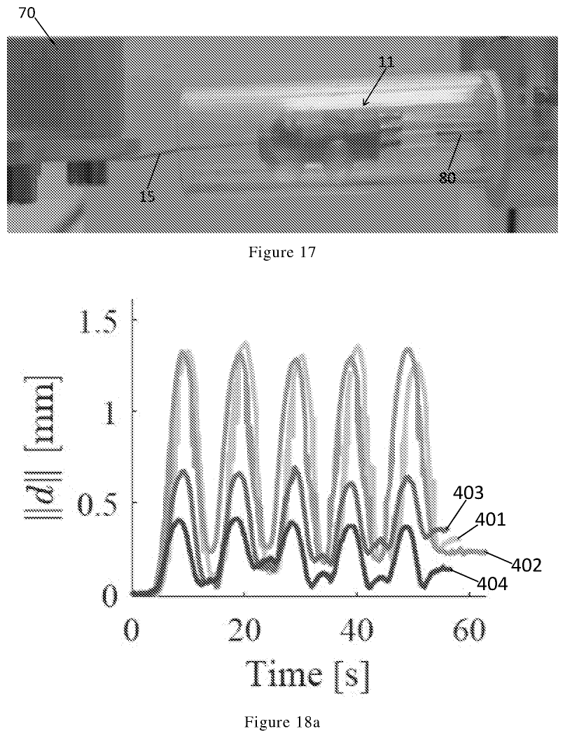

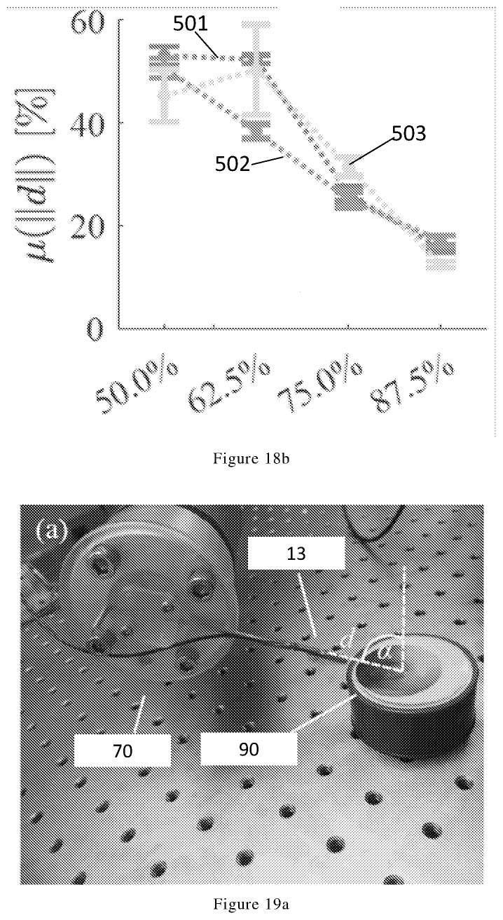

FIG. 17 shows an experimental set-up used to test the motion-compensation capabilities of the system. The set-up comprises a prototype positioning device 11 arranged within a tube representing an ear canal, an electromagnetic tracker 80, a needle 15 and a robotic manipulator 70 configured to be controlled to manipulate the needle 15. The robotic manipulator 70 represents a user's hand, which may be subject to undesired motion, and the electromagnetic tracker 80 represents the end of the needle 15 to be inserted into the tympanic membrane. The robotic manipulator 70 was used to generate a needle displacement of 2.5 mm from an initial position, which was repeated five times. The electromagnetic tracker 80 was used to track the response of the system for four different inflation volumes of the actuators: 50%, 62.5%, 75% and 87.5% of maximum volume. The resulting motion recorded by the electromagnetic tracker 80 for each inflation volume for the EF30 prototype is shown in FIG. 18a. Line 401 shows the resulting motion recorded by the electromagnetic tracker 80 for 50% of maximum inflation volume, line 402 for 62.5%, line 403 for 75% and line 404 for 87/5%. FIG. 18b shows mean peak displacement for each inflation volume for each the EF30, EF50 and DSFX prototypes (lines 501, 502 and 503 respectively).

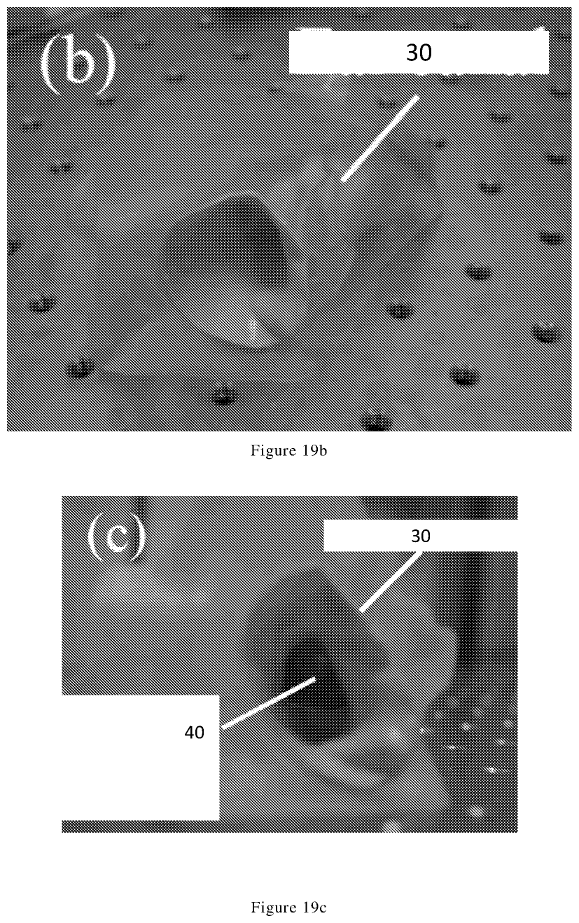

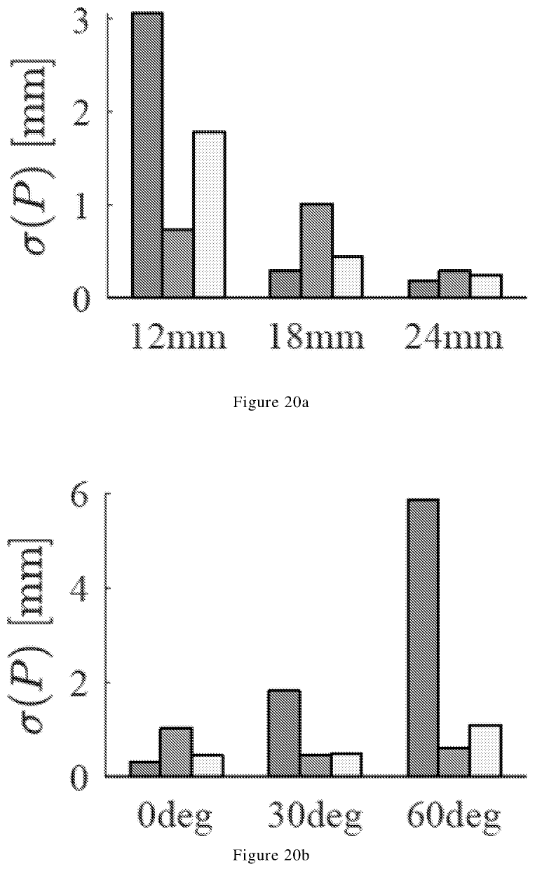

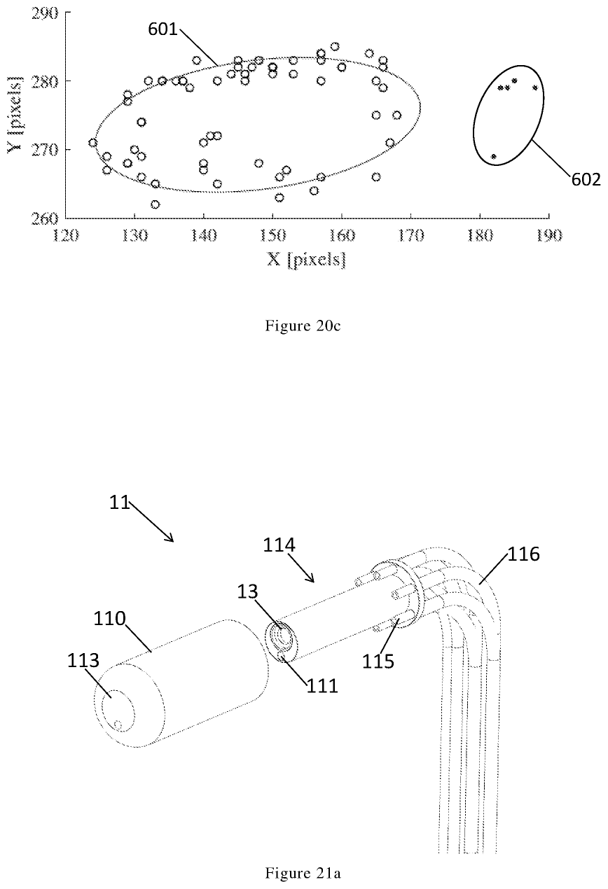

FIG. 19a shows an experimental set-up used to test the image recognition capabilities of the system. The experimental set-up includes a camera 13, a robotic manipulator 70 configured to control movement of the camera 13, and a test image 90 representative of an image captured by the camera of the system in use. Images were captured at various distances ‘d’ and various angles of incidence ‘a’ from the test image 90. Three different test images representing increasing degrees of image recognition difficulty were used. The results were validated using a phantom ear canal 30 within which an image of a tympanic membrane 40 was positioned (as shown in FIGS. 19b and 19c). FIG. 20a shows the recorded standard deviation ‘o’ of the target location identified by the system for each of the three test images at distances of 12 mm, 18 mm and 24 mm between the camera and the respective test image and at an angle of incidence of 0 degrees. The three images were selected in terms of the predicted difficulty encountered by the system in identifying the target location within the respect image according to a qualitative preliminary evaluation. The three images were rated in terms of difficulty as ‘high’, ‘medium’ and ‘low’. For each distance between the camera and the respective test image, the left-hand bar in FIG. 20a represents the standard deviation ‘o’ of the target location identified by the system for the ‘high’ difficulty test image, the middle bar represents the standard deviation ‘o’ for the ‘medium’ difficulty image, and the right-hand bar represents the standard deviation ‘o’ for the ‘low’ difficulty image. FIG. 20b shows the same results as FIG. 20a, but for angles of incidence of 0, 30 and 60 degrees and at a distance of 18 mm between the camera and the respective test image.

In order to test the performance of the system in identifying a target location within an image, a prototype positioning device was inserted into a phantom ear canal 30 within which an image of a tympanic membrane 40 was positioned (as shown in FIGS. 19b and 19c). The actuators of the positioning device were then operated to move the cylindrical body in a circular motion, similar to that represented in FIG. 16d, while images were captured using the camera. FIG. 20c shows a plot of the position of the target location identified by the system in each of the captured images. Line 601 shows that the identified target locations follow an elliptical path as the cylindrical body was moved in a circular motion. Outlier target locations are circled by line 602.

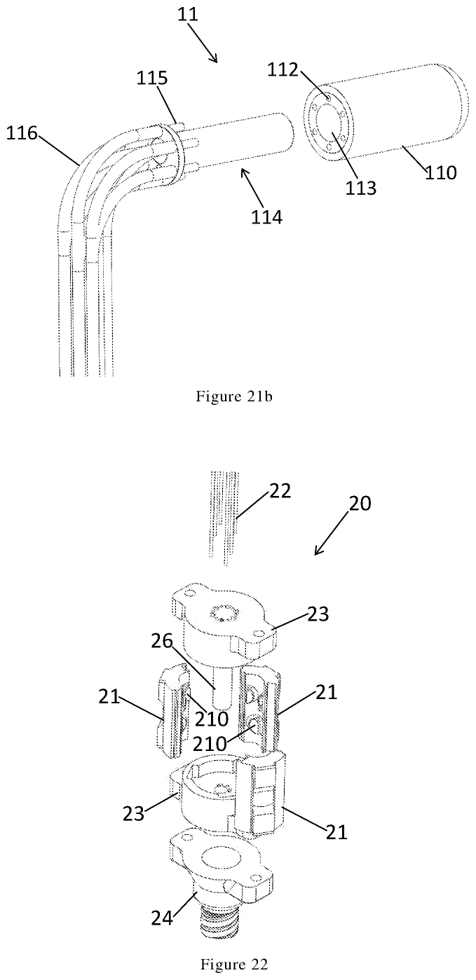

FIGS. 21a and 21b show a schematic view of a positioning device 11 of a system according to another embodiment of the invention. FIG. 21a shows a front isometric view of the positioning device 11 and FIG. 21b shows a rear isometric view of the positioning device 11. The positioning device 11 of FIGS. 21a and 21b shares features in common with the positioning device 11 of FIGS. 2a to 2c and like reference numerals are used to refer to like features. For clarity, not all the features of the positioning device 11 are shown in FIGS. 21a and 21b. The positioning device 11 of FIGS. 21a and 21b may be used in the system 10 of FIG. 1 or the system 100 of FIG. 5.

In the embodiment of FIGS. 21a and 21b, the cylindrical body 110 comprises a disposable element. As in the embodiment of FIGS. 2a to 2c, the cylindrical body 110 comprises a core, a membrane and six actuators in the form of inflatable chambers (not shown in FIGS. 21a and 21b). The cylindrical body 110 also comprises six inflation channels 112, as shown in FIG. 21b, each inflation channel 112 being configured to supply an inflation fluid to one of the actuators. The core of the cylindrical body 110 of FIGS. 21a and 21b further comprises a longitudinal bore 113.

The positioning device 11 of FIGS. 21a and 21b further comprises a reusable insert 114 comprising a camera 13, a longitudinal bore 111 for receiving a needle and six inflation tubes 115. The longitudinal bore 111 is formed within the main body of the reusable insert, which also provides a housing for the camera 13. In use, each inflation tube 115 is connected to a fluid line 116 for supplying and inflation fluid to the respective inflation tube 115. The fluid lines 116 may be formed from silicone or another suitably flexible material so as not to restrict positioning of the positioning device 11.

The longitudinal bore 113 of the core of the cylindrical body 110 is configured to receive the body of the reusable insert 114 and the inflation channels 112 are each configured to receive a different one of the inflation tubes 115. An interference fit is provided between the core of the cylindrical body 110 and the reusable insert 114 and a fluid-tight connection is provided between the inflation channels 112 and the inflation tubes 115. Once the reusable insert 114 is installed within the cylindrical body 110, the positioning device 11 is operable in the same way as the positioning device 11 of FIGS. 2a to 2c. After the positioning device 11 has been utilised, for example to perform an intratympanic injection, the cylindrical body 110 can be removed from the reusable insert 114 and disposed of. A new cylindrical body 110 can then be installed on the reusable insert 114 for the next use.

FIG. 22 shows a schematic representation of a moulding apparatus 20 for producing the cylindrical body 110 of the positioning device 11 of FIGS. 21a and 21b. The moulding apparatus 20 of FIG. 22 shares features in common with the moulding apparatus 20 of FIGS. 4a-c and like reference numerals are used to refer to like features. The moulding apparatus 20 of FIG. 22 differs from the moulding apparatus 20 of FIGS. 4a-c in that the round bar 25 is replaced with a projection 26 protruding into the moulding volume from the endcap 23 corresponding to the rear of the positioning device 11. The projection 26 is provided to form the longitudinal bore 113 of the core of the cylindrical body 110. It will be appreciated that a similar moulding apparatus 20 may be used to produce the cylindrical body 110 of the positioning device 11 of FIGS. 2a to 2c, with the projection 26 provided to form the channel to receive the camera 13 or optical fibres.

Although specific examples have been described, the skilled person will appreciate that variations are possible, within the scope of the invention, which should be determined with reference to the accompanying claims.

What is claimed is:

1. A system for positioning a medical tool within a lumen, comprising:
a positioning device comprising:
a cylindrical body configured to receive the medical tool; and
an actuator configured to apply a force to an internal wall of the lumen to move the cylindrical body in a radial direction within the lumen;
and
a control system configured to: receive an input signal indicative of a target position of the cylindrical body, and provide an output signal to control the actuator to move the cylindrical body to adopt the target position.

2. The system of claim 1, wherein the target position comprises a radial position of a longitudinal axis of the cylindrical body relative to a longitudinal axis of the lumen, and an angular orientation of the longitudinal axis of the cylindrical body relative to the longitudinal axis of the lumen.

3. The system of claim 1, wherein the control system is configured to:
i) automatically obtain the input signal indicative of a target position of the cylindrical body; and/or ii) automatically provide the output signal to control the actuator to move the cylindrical body to adopt the target position.

4. (canceled)

5. The system of claim 1, wherein the system comprises a camera or optical fibres, and the control system is configured to process an image captured by the camera or optical fibres to produce the input signal indicative of the target position.

6. The system of claim 5, wherein the control system is configured to process the image using an artificial neural network trained to identify a target location in the image and produce the input signal indicative of the target position of the cylindrical body in dependence on the target location in the image.

7. (canceled)

8. The system of claim 1, wherein the control system is configured to:
i) receive a further input signal indicative of a deviation of the cylindrical body from the target position; and/or- ii) provide a further output signal to control the actuator to move the cylindrical body to adopt the target position in response to the further input signal.

9. (canceled)

10. The system of claim 1, wherein the actuator comprises an inflatable actuator or a liquid inflatable actuator.

11. The system of claim 10, comprising a positive displacement pump configured to deliver a fluid to the inflatable actuator to inflate the actuator.

12. The system of claim 11, wherein the output signal to control the actuator is indicative of a target volume for the inflatable actuator.

13. (canceled)

14. The system of claim 10, wherein the cylindrical body comprises a core and an outer membrane arranged on an external surface of the core and the actuator comprises a portion of the outer membrane.

15. (canceled)

16. The system of claim 1, wherein the force is offset from the centre of the cylindrical body.

17. The system of claim 1, wherein the positioning device comprises a plurality of actuators and each actuator is configured to apply a radial force to an internal wall of the lumen to move the cylindrical body in a different radial direction.

18. The system of claim 17, wherein a first one of the plurality of actuators is located at a first longitudinal position of the cylindrical body and a second one of the plurality of actuators is located at a second longitudinal position of the cylindrical body.

19. The system of claim 1, wherein the positioning device comprises a first plurality of actuators located at a first longitudinal position of the cylindrical body and a second plurality of actuators located at a second longitudinal position of the cylindrical body.

20. The system of claim 1, wherein the output signal to control the actuator comprises a pressure to be applied by the actuator to the internal wall of the lumen.

21. The system of claim 1, wherein the medical tool comprises a needle.

22. The system of claim 21, wherein the cylindrical body of the positioning device comprises a longitudinal bore for receiving the needle.

23. The system of claim 1, wherein the lumen comprises an ear canal.

24. The system of claim 23, wherein the target position of the cylindrical body is aligned with the round window of a cochlea.

25. The system of claim 24, wherein a target location in the image is the centre of the posteroinferior quadrant of the tympanic membrane.
